(19) United States
(12) Patent Application Publication
(10) Pub. No.: US 2018/0251574 A1
(43) Pub. Date: Sep. 6, 2018

(54) FUCAN-DENDRIMER BASED COMPOUNDS AND COMPLEXES

(71) Applicant: ARC Medical Devices Inc., Richmond (CA)

(72) Inventor: Christopher Michael Kevin Springate, Surrey (CA)

(73) Assignee: ARC Medical Devices Inc., Richmond (CA)

(21) Appl. No.: 15/970,472

(22) Filed: May 3, 2018

Related U.S. Application Data
(60) Provisional application No. 62/665,976, filed on May 2, 2018.

(51) Int. Cl. C08B 37/00; A61K 47/36; A61K 47/10

(52) U.S. Cl. CPC C08B 37/0006; A61K 47/10; A61K 47/36

(57) ABSTRACT

Compositions, systems, methods, etc., related to fucan-dendrimer complexes wherein a fucan molecule is linked or otherwise attached to a dendrimer. For example, the current compositions can comprise fucan covalently linked, conjugated, attached, tagged, adducted, bound, etc., to a dendrimer. These fucan-dendrimer complexes include fucan-hyperbranched polyglycerol, fucan-hyperbranched poly(glycerol ester), fucan-hyperbranched poly(1,3-diether), fucan-poly(3-ethyl-3-(hydroxymethyl)oxetane), fucan-hyperbranched polyesters, fucan-hyperbranched polyethylene, fucan-hyperbranched polystyrene, fucan-hyperbranched poly(urea-urethanes), fucan-hyperbranched polyethyleneimine, fucan-hyperbranched poly(amido amine)s, fucan-hyperbranched polyphosphates, fucan-hyperbranched polypeptides, fucan-hyperbranched polysaccharides, fucan-hyperbranched polyacrylates and fucan-hyperbranched beta-cyclodextrin. These fucan-dendrimer complexes may be used in medically and/or pharmaceutically effective compositions, for a plurality of applications, including the treatment of fibrous adhesions. In some embodiments, the fucan is fucoidan.

### BACKGROUND

Fucans (including fucoidan) are sulfated polysaccharides. In general terms, this means that they are molecules made up of a number of sugar groups, and also have sulfur atoms attached to the sugar groups. The main sugar group is called “fucose”, which is sugar that has 6 carbon atoms and has the chemical formula C₆H₁₂O₅. “Fucoidan” (or fucoidin) indicates fucans derived from brown algae (seaweed). Fucans can exist alone, or in a mixture of other sugars, for example in a mixture of sugars such as xylose, galactose, glucose, glucuronic acid and/or mannose. These other sugars may be extracted from the seaweed or other source with the fucan. Although fucans are currently derived from natural sources such as the brown algae (seaweeds), sea cucumbers, etc., mentioned herein, “fucan” includes polymer molecules having the chemical and structural motifs of the fucans as discussed herein regardless of the ultimate source(s) of the fucans.

Fucoidan can be obtained from a variety of species of brown algae including but not limited to: Adenocystis utricularis, Ascophyllum nodosum, Chorda filum, Cystoseirabies marina, Durvillaea antarctica, Ecklonia kurome, Ecklonia maxima, Eisenia bicyclis, Fucus evanescens, Fucus vesiculosis, Hizikia fusiforme, Himanthalia Elongata, Kjellmaniella crassifolia, Laminaria brasiliensis, Laminaria cichorioides, Laminaria hyperborea, Laminaria japonica, Laminaria saccharina, Lessonia trabeculata, Macrocystis pyrifera, Pelvetia fastigiata, Pelvetia canaliculata, Saccharina japonica, Saccharina latissima, Sargassum stenophylum, Sargassum thunbergii, Sargassum confusum, Sargassum fusiforme and Undaria pinnatifida. These exemplary species are all from the taxonomic class Phaeophyceae and the majority of these species fall into the families of Fucales and Laminariaceae.

Thus, there has gone an unmet need for fucan-dendrimer complexes that can provide an expansion in the suitability of fucan in or for several potential applications and/or provide for an expansion in the suitability of dendrimers in or for several potential applications. Fucan-dendrimer complexes may lead, for example, to an increase in surfactant properties of the fucan, to an increased solubility of fucan where fucan is desired in low volume or for high concentration purposes, may facilitate mobility of the fucan to target sites, and/or may result in the reduction of the amount fucan required for a particular efficacy for a particular purpose such as treatment of a particular disease or condition. The present compositions, methods, etc., provide these and/or other advantages.

### SUMMARY

The current compositions, systems, methods, etc., herein relate to fucan-dendrimer complexes that expand the suitability of using fucan in or for several potential applications including medical treatments. The fucan-dendrimer complexes herein comprise a fucan molecule linked to a dendritic molecule. The combination, i.e., fucan-dendrimer complex, can be effective for such treatments, etc., for example by providing the advantageous features provided by fucans coupled with the advantageous features provided by the dendritic component.

In certain embodiments, the current compositions comprise fucan conjugated, attached, tagged, adducted, linked, etc., to the dendritic molecule. The attachment can be, e.g., through at least one of a covalent bond, ionic bond, adsorption, dipole-dipole interaction, hydrogen bonding, co-ordination complex formation or magnetic interaction. These fucan-dendrimer complexes include fucan-hyperbranched polyglycerol (HPG), fucan-hyperbranched poly(glycerol ester), fucan-hyperbranched poly(1,3-diether), fucan-poly(3-ethyl-3-(hydroxymethyl)oxetane), fucan-hyperbranched polyesters, fucan- hyperbranched polyethylene, fucan-hyperbranched polystyrene, fucan-hyperbranched poly(urea-urethanes), fucan-hyperbranched polyethyleneimine, fucan-hyperbranched poly(amido amine)s, fucan-hyperbranched polyphosphates, fucan-hyperbranched polypeptides, fucan-hyperbranched polysaccharides, fucan-hyperbranched polyacrylates and fucan-hyperbranched beta-cyclodextrin. These fucan-dendrimer complexes may be used for a plurality of applications, including the treatment of fibrous adhesions. In some embodiments, the fucan is fucoidan.

For example, this can include a fucan-dendrimer complex can comprise a fucan molecule attached to a hyperbranched polyglycerol as the secondary component.

The hyperbranched polyglycerol can itself comprise a further substituent; the substituent on the hyperbranched polyglycerol can comprise at least one of amine, ammonium, thiol, sulfonic acid, phosphonic acid, carboxylic acid, tosyl, carboxyl, hydroxyl, n-hydroxysuccinimide, methoxy polyethylene glycol, polyethylene glycol, aryl groups, alkyl groups. The hyperbranched polyglycerol can further can comprise a co-polymer, which can for example can be at least one of polyglycerol, a polyol, a polyalkylene glycol, a polyalkylene glycol-alkyl ether, or an alkyl chain. The hyperbranched polyglycerol can be polymerized from at least one of a C₁-C₂₀ alkyl epoxide, C₁-C₂₀ alkyl glycidyl ether, glycerol epoxide, C₁-C₂₀ alkyl, or can be polymerized from a C₁-C₂₀ alkyl substituted with at least one OR group where R can be one of a hydrogen, a cationic moiety, or a polymer segment.

The hyperbranched polyglycerol can have a number average molecular weight between 1-100,000 kDa and can have a PDI of between about 1.0-3.5. The hyperbranched polyglycerol can comprise a biologically active moiety, which can be at least one of valrubicin, cisplatin, paclitaxel, docetaxel, mitomycin, vinblastine, methotrexate, doxorubicin, or suramin, including analogs, salts, etc., of such biologically active moieties.

The fucan can be fucoidan, and the fucan-dendrimer complex can be contained within a medical device, combination product or pharmaceutically acceptable and/or therapeutically active medical composition.

These and other aspects, features and embodiments are set forth within this application, including the following detailed description. Unless expressly stated otherwise, all embodiments, aspects, features, etc., can be mixed and matched, combined and permuted in any desired manner. Various references are set forth herein, including in the Cross-Reference To Related Applications and List Of References, that discuss certain compositions, secondary components, systems, apparatus, methods and other information; all such references are incorporated herein by reference in their entirety and for all their teachings and disclosures, regardless of where the references may appear in this application. Citation to a reference herein is not an admission that such reference constitutes prior art to the current application.

### DETAILED DESCRIPTION

The current compositions, systems, methods, etc., herein comprise fucan-dendrimer complexes comprising a fucan molecule linked to a dendrimer. The fucan-dendrimer complexes can be effective for medical treatments, disease inhibition etc. In certain embodiments, the current fucan-dendrimer complexes comprise fucan covalently bound, ionically bound, hydrogen-bonded, co-ordinated, magnetically bound, conjugated, attached, tagged, adducted, linked, etc., to the dendritic molecule. These fucan-dendrimer complexes include fucan-hyperbranched polyglycerol (HPG), fucan-hyperbranched poly(glycerol ester), fucan-hyperbranched poly(1,3-diether), fucan-hyper branched poly(3-ethyl-3-(hydroxymethyl)oxetane), fucan-hyperbranched polyesters, fucan-hyperbranched polyethylene, fucan-hyperbranched polystyrene, fucan-hyperbranched poly(urea-urethanes), fucan-hyperbranched polyethyleneimine, fucan-hyperbranched poly(amido amine)s, fucan-hyperbranched polyphosphates, fucan-hyperbranched polypeptides, fucan-hyperbranched polysaccharides, fucan-hyperbranched polyacrylates and fucan-hyperbranched beta-cyclodextrin. These fucan-dendrimer complexes may be used for a plurality of applications, including the treatment of fibrous adhesions. In some embodiments, the fucan is fucoidan. The current fucan-dendrimer complexes can themselves be, or can be included on or in, medical devices, combination products or in pharmaceutically acceptable, therapeutically and/or medically effective compositions.

The fucan-dendrimer complexes herein may be used for a plurality of applications, including the treatment of fibrous adhesions and other targets such as other diseases and/or conditions. Treatment indicates that the fucan-dendrimer complex reduces or prevents the development of a target disease or other condition, such as reducing or preventing the formation of fibrous adhesions at a target site, and also includes elimination of existing diseases or other conditions, including for example the elimination of already-existing fibrous adhesions. For such treatment, the fucan-dendrimer complex is typically provided in a medically acceptable, medical device, combination product or pharmaceutically effective composition that contains additional components such as binders, adjuvants, excipients, etc., as well as, if desired additional medically active substances such as secondary drugs that are contained within the composition but not attached to the fucan or fucan-dendrimer complex.

In some embodiments, the fucan is fucoidan.

“Fucan” is used herein consistent with its ordinary meaning. For example, fucan comprises a polymeric structure composed of monomeric repeats of monosaccharide units represented by formula 1a, formula 1b and/or formula 1c. The repeating element in the fucan polymer comprising Formula 1a must include fucose and can also include other hexose deoxy saccharide monomers (exemplified below by fucose); Formula 1b may be any hexose saccharide monomer (exemplified below by galactose saccharide monomer); Formula 1c is any uronic acid (exemplified below by glucuronic acid monomer).

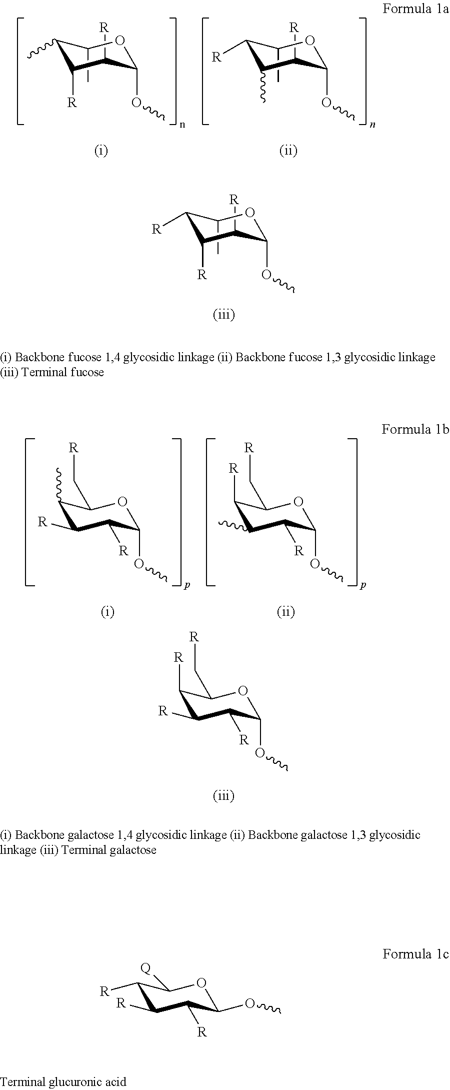

The monomeric repeats depicted in formula 1a may be linked to monomeric repeats depicted in formula 1a, formula 1b or formula 1c. The monomeric repeats depicted in formula 1b may be linked to monomeric repeats depicted in formula 1a, formula 1b or formula 1c. In the formula above, n and/or p may be between about 5 and 100,000+.

Continuing to refer to the molecular structure diagrams above, R is an appropriate substituent according to the given purpose discussed herein for a particular embodiment, for example R is selected from the group consisting of Formula 1a, Formula 1b, Formula 1c (i.e., to provide polymers), —H, —OH, —OS(O)(O)O⁻, —OS(O)(O)OR¹, —OP(O)(OH)₂, —OP(O)(OH)OR¹), —OP(O)(OR¹)₂ X, —OR¹, —R¹, ═CH—R¹, ═CR¹R¹, —O(O)CR¹, S(O)R¹, S(O)(O)R¹, —S(O)NHR¹, S(O)NR¹R¹, —NHR¹NR¹R¹, NHC(O)R¹, —NR¹C(O)R¹, —S—R¹, —SSR¹, NHC(O)OR¹, NR¹C(O)OR¹, NHC(O)NHR¹, NHC(O)NR¹R¹, NR¹C(O)NHR¹, NR¹C(O)NR¹R¹, —CN, COOH, —COOR¹, NO₂, NH₂, NHOR¹, N₃, C₃₋₆ carbocycle, C₃₋₆ substituted carbocycle where the substitution may be selected independently from one or more R¹ groups, heterocycle, or a substituted heterocycle where the substitution may be selected independently from one or more R¹ groups. Where two R¹ groups are joined to a single atom they may be independently selected from the group of R¹ constituents or moieties.

Continuing to refer to the molecular structure diagrams above, Q is a suitable substituent according to the given purpose discussed herein for a particular embodiment. For example, Q can be selected from the group consisting of —COOH, —COOR¹, —CONHR¹, CONR¹R¹, —X, —CH₂OR¹, —CH₂X, CSNHR¹, CSNR¹R¹, C(S)OR¹, —CH₂NH₂, —CH₂NHR¹, —CH₂N(R¹)₂, —CH₂NHS(O)(O)R¹.

For both R and Q, where R¹ is selected from the group consisting of —H, —X, C₁₋₃₀ alkyl, C₁-C₃₀ allyl, C₁₋₃₀ alkenyl, where any and all hydrogens may be independently substituted with X, C₃₋₆ carbocycle and heterocycle, R²Y,

R² can be, for example, selected from the group comprising —H, —X, C₁₋₃₀ alkyl, C₁-C₃₀ allyl, C₁₋₃₀ alkenyl where any and all hydrogens may be independently substituted with X, C₃₋₆ carbocycle and heterocycle.

X can be, for example, F, Cl, Br, I or OH.

Y can be a terminal functional group or terminal functional linking group selected from, —OH, —O-(L), —S-(L), —SS-(L), —OS(O)(O)O⁻, X, SH, NH₂, —NH-(L), —N-(L)₂, NO₂, CN, C(O)OH, —C(O)O-(L), —OC(O)-(L) —CONH₂, —CONH-(L), —C(O)N-(L)₂, —NHC(O)-(L), —S(O)(O)NH-(L), —S(O)(O)N-(L)₂, NHS(O)(O)-(L), —OC(O)NH-(L), —OC(O)N-(L)₂, —NHC(O)O-(L), C₅₋₆ heterocycle-L, C₃₋₆carbocycle-L, with L herein depicting a dendrimer, exemplified by, but not limited to hyper branched polyglycerols (HPG).

The dendrimer may be attached covalently to fucan through synthetic chemistry methods, including for example known methods to form a covalent linkage via an appropriate functionality located on the dendrimer. Appropriate functionalities include but are not limited to primary amines, secondary amines, carboxylic acid (and reactive derivatives thereof), sulfuryl halides, hydroxyls, thiols and halogens.

The fucan and dendrimer are the primary components of the fucan-dendrimer complex. Further to, or instead of covalent linkage between the two components, the fucan and dendrimer may also be bound to each other by additional or alternative methods that may include but are not limited to ionic bonding, adsorption, dipole-dipole interactions, hydrogen bonding, co-ordination complex formation and magnetic interactions. The stoichiometric ratio of fucan to dendrimer may range from about 1:0.001 to 1:100,000, typically between about 1:0.01 to 1:20,000, or between 1:0.1 to 1:10,000 or 1:1 to 1:100.

The fucan-dendrimer complexes discussed herein, along with related methods, systems, compositions, medical compositions, etc., may provide advantages over non-complexed constituents of the fucan-dendrimer complexes. The advantages may include a combination of attributes related to the fucan and the dendrimer it is complexed with. Fucans, for example, have been shown to be efficacious in the treatment of fibrous adhesions, and the dendrimer may assist in time-controlled release of the fucan at a fibrous adhesion target site. Indeed, the dendrimer may assist in time-controlled release of the fucan at any desired target site.

In some embodiments, the fucan-dendrimer complexes discussed herein utilize the fucan at a targeted physiologically relevant site such as a disease site or the location of a targeted condition such as a surgical site or an injury site.

The fucan-dendrimer complexes herein can further comprise a further physiologically effective agent attached to the dendrimer. Such further physiologically effective agent can be, for example, a drug, prodrug, or other physiologically effective compound or molecule. Such further physiologically effective agent is directed against the target site, i.e., provides a medically beneficial effect at the target site. For example, in some embodiments, the further physiologically effective agent in the fucan-dendrimer complex can be a beta-blocker and the overall complex can be configured for the treatment of heart diseases.

Fucan-dendrimer complexes discussed herein can also aid in the solubility of the fucan as desired, for example to maintain or achieve high concentration. The concentration achieved may be between 100-850 mg/mL and for example between 300-700 mg/ml. The dendrimer may be a hyperbranched polyglycerol. One or more of the hydroxyl groups on the sugar monomers of the fucan may be functionalized.

Where the dendrimer may be mucoadhesive and help the fucan-dendrimer complex adhere to tissue. The fucan-dendrimer complex may have greater surfactant or lipophilic properties than the fucan and may have better penetration through tissues. For example, when applied topically the fucan-dendrimer complex may be absorbed into and through the skin to treat skin conditions such as keloid trait scars or enter the systemic circulation; and when taken orally may result in greater bioavailability than is found with fucan alone.

Other fucan-dendrimer complexes discussed herein, including for example fucan-HPG, control the release of the fucan in the desired application. For example, hydrogen bonding between fucoidan and HPG may increase the viscosity of the solution and reduce the rate at which fucoidan-HPG is removed from the target site. The viscosity of the solution may be increased by for example 25-200%. The rate of release of the fucan at the target site may be increased by between 1-300 days, for example between 1-29 days. The target site may be the abdominal cavity in the case of an abdominal adhesion. The fucan-HPG complex may also provide a controlled release of the moiety attached to the fucan in certain applications where desired.

The fucan-dendrimer complexes discussed herein are not intended to be limited to a single advantage mentioned. It is to be understood that any fucan-dendrimer complex discussed herein may result in advantageous attributes beyond a primary desired advantage of the fucan-dendrimer complex. A fucan-dendrimer complex may be linked to an additional moiety or moieties in succession, for example to produce a fucan-HPG-paclitaxel complex.

In a further aspect, the present compositions, etc., are themselves or are provided with medical devices or surgical devices suitable for implantation in, or other administration to, a patient. Suitable surgical devices can be coated in or made of a fucan-dendrimer complex composition as discussed herein. The surgical device can be for example a film, membrane, stent, catheter, port, shunt, device for continuous subarachnoid infusion, feeding tube, solid implant to prevent surgical adhesion, uterine implant, artificial sphincter, periurethral implant, splint, ophthalmic implant, contact lens, plastic surgery implant or other device as desired. A suitable stent can be an esophageal stent, gastrointestinal stent, vascular stent, biliary stent, colonic stent, pancreatic stent, ureteric stent, urethral stent, lacrimal stent, Eustachian tube stent, fallopian tube stent, nasal stent, sinus stents, tracheal stent, or bronchial stent. The surgical device can also be a venous access device comprising an external tunneled catheter, implanted port, epidural catheter or central catheter (PICC).

In still another further aspect, the present discussion provides kits comprising a composition as discussed herein in a medical device, combination product or pharmaceutically acceptable container, such as a syringe or a vial. The kits can comprise a surgical device as discussed herein in a medical device, combination product or pharmaceutically acceptable container. The kits can further comprise a notice associated with the container, the notice typically in a form prescribed by a governing agency regulating the composition, and can further comprise instructions about at least one of use of the composition, dosing a patient and mode of administration.

### Additional Discussion of Certain Exemplary Diseases.

A fibrous adhesion is a type of scar that forms between two parts of the body, usually after surgery (surgical adhesion). Fibrous adhesions can cause severe problems. For example, fibrous adhesions involving the female reproductive organs (ovaries, Fallopian tubes) can cause infertility, dyspareunia and severe pelvic pain. Fibrous adhesions that occur in the bowel can cause bowel obstruction or blockage, and fibrous adhesions can also form in other places such as around the heart, spine and in the hand. In addition to surgery, fibrous adhesions can be caused for example by endometriosis, infection, chemotherapy, radiation, trauma and cancer.

Terms such as surgical adhesions, post-surgical adhesions, postoperative adhesions, adhesions due to pelvic inflammatory disease, adhesions due to mechanical injury, adhesions due to radiation, adhesions due to radiation treatment, adhesions due to trauma, and adhesions due to presence of foreign material all refer to adherence of tissues to each other due to a similar mechanism and are all included in the term fibrous adhesions.

Various attempts have been made to prevent surgical adhesions. These have involved pharmacological approaches targeted at influencing the biochemical and cellular events that accompany surgical traumas well as barrier methods for the separation of affected tissues. For example, the use of peritoneal lavage, heparinized solutions, procoagulants, modification of surgical techniques such as the use of microscopic or laparoscopic surgical techniques, the elimination of talc from surgical gloves, the use of smaller sutures and the use of physical barriers (films, gels or solutions) aiming to minimize apposition of serosal surfaces, have all been attempted. Preventive therapies have also included prevention of fibrin deposition, reduction of inflammation via anti-inflammatory drugs, and removal of fibrin deposits. Therapies have also included the administration of fucan (without complexed dendrimers) to a target site.

Interventional attempts to prevent the formation of post-surgical adhesions have included the use of hydroflotation techniques or barrier devices. Hydroflotation involves the instillation of large volumes of polymer solutions such as dextran, or carboxymethyl cellulose into the surgical space in an attempt to keep the organs apart. Synthetic barrier membranes made from oxidized regenerated cellulose (for example Interceed™), polytetrafluoroethylene (Gore-Tex surgical membrane), and fully resorbable membranes made from a modified hyaluronic acid/carboxymethylcellulose (HA/CMC) combination (for example Serafima™) have also been used to reduce post-surgical adhesion formation in both animals and humans. The success of these HA/CMC membranes may derive from their ability to provide tissue separation during the peritoneal wound repair process when fibrous adhesions form. Unfortunately, limited success has been seen with these methods.

Cancers Generally:

Cancer has been the second leading cause of death in the U.S. and accounts for over 20% of all mortalities. Cancer is a proliferative disease and is characterized by the uncontrolled division of certain cells, which may lead to the formation of one or more tumors. A number of methods are used to treat cancer, including surgery, radiation, chemotherapy and combinations thereof. Although surgery is a relatively common method used for some localized tumors, there is still a significant chance of tumor recurrence after tumor excision.

Treating cancers and other proliferative diseases has been limited by the potential for damage or toxicity to non-cancerous, healthy tissues. In radiation and surgical treatments, the procedure has been generally confined to and proximal to the tumor sites. However, there can be significant risk to patients undergoing surgical removal of cancerous tissues (e.g., in removal of prostate or brain tumors there can be a significant risk of non-repairable damage to surrounding vital tissues, for example via potential reduced need for resection of non-tumor tissues. Furthermore, in focused radiation treatment, which has been given as a first line treatment for prostate cancer, there are similar risks. In the chemotherapeutic treatment of cancer, the drug has been administered systemically, so that the whole body is exposed to the drug. These drugs are designed to be toxic to cancer cells, but they are also (generally) toxic to non-cancerous cells so that patients become quite ill when undergoing drug treatments for cancer. Through experience, oncologists are able to give doses of these drugs that may be tolerated by some patients. However, these doses are often not successful in treating cancers.

One problem with any method of treating cancer has been the local recurrence of the disease. For example, approximately 700,000 Americans are diagnosed with localized cancer annually (approximately 64% of all cancer patients) and almost half a million are treated using surgical methods. Unfortunately, 32% of patients treated with surgery relapse after the initial treatment (approximately 21% relapse at the initial surgical site and 11% at distant metastatic sites). Almost 100,000 patients die annually due to localized recurrence of cancer. This has been especially true in breast cancer where 39% of patients undergoing lumpectomy will experience local recurrence of the disease.

Staging is a method of judging the progress of the cancer (solid tumor) in a patient. A simplified approach puts patients into three groups or stages based on how far the cancer has advanced:

Stage 1: The cancer can be treated by surgically removing part of the organ. This is also known as the resectable stage.

Stage 2: The cancer has advanced past the point of being resectable, but is still confined to the organ itself.

Stage 3: The tumor has spread to other organs.

Many cancers are treated with anti-proliferative agents including, for example, 5-fluorouracil (Efudex®), vinca alkaloids (for example, vincristine (Oncovin®)), anthracyclines (for example, doxorubicin (Adriamycin®)), cisplatin (Platinol-AQ®), gemcitabine hydrochloride (Gemzar®), methotrexate and paclitaxel. Some examples of the toxicities associated with the anti-proliferative agents, methotrexate and paclitaxel, are discussed elsewhere herein. Methotrexate has been used to treat several cancers including, for example, bladder, breast, cervical, head and neck, hepatic, lung, and testicular cancers. Paclitaxel has been used to treat several cancers including, for example, ovarian, breast, and non-small cell lung cancers. Compendium of Pharmaceutical and Specialties Thirty-fifth Edition (2000).

Toxicities due to 5-fluorouracil can include cardiovascular toxicity such as myocardial ischemia; central nervous system toxicities such as euphoria, acute cerebellar syndrome and ataxia; dermatologic toxicities such as alopecia and dermatitis; gastrointestinal toxicities such as nausea, vomiting and oral or gastrointestinal ulceration; hematologic toxicities such as leukopenia, thrombocytopenia and anemia; hypersensitivity toxicities such as anaphylaxis and contact hypersensitivity; ocular toxicities such as increased lacrimation, photophobia and conjunctivitis; and, other toxicities such as fever. 5-fluorouracil has been used to treat many cancers including, for example, breast, colorectal, gastric, hepatic, bladder, head and neck, non-small cell lung, ovarian, pancreatic, and prostate cancers. Compendium of Pharmaceutical and Specialties Thirty-fifth Edition (2000).

Toxicities due to vincristine include central nervous system toxicities such as seizures in children and hallucinations; dermatologic toxicity such as alopecia; extravasation toxicity such as vesicant; gastrointestinal toxicities such as nausea, vomiting, constipation and stomatitis; hematologic toxicity such as myelosuppression; neurologic toxicities such as peripheral neuropathy and autonomic neurophathy; ocular toxicities such as double vision, transient blindness and optic atrophy; renal/metabolic toxicities such as urinary retention, hyperuricemia and bladder atony; respiratory toxicity such as shortness of breath; and, other toxicity such as fever in children. This anti-proliferative agent has been used to treat several cancers including, for example, Hodgkin's disease, small cell lung, Wilm's tumor, and testicular cancers. Compendium of Pharmaceutical and Specialties Thirty-fifth Edition (2000)

Toxicities due to doxorubicin include cardiovascular toxicities such as electrocardiographic abnormalities and cardiomyopathy; dermatologic toxicities such as alopecia and nail changes; extravasation hazard toxicity such as vesicant; gastrointestinal toxicities such and nausea, vomiting and stomatitis; genitourinary toxicity such as red coloration of urine; hematologic toxicity such as myelosuppression; hypersensitivity toxicities such as anaphylaxis and skin rash; ocular toxicity such as conjunctivitis; reproductive toxicity such as infertility; and, other toxicity such as hyperuricemia. This anti-proliferative agent has been used to treat several cancer including, for example, breast, small cell lung, and ovarian cancers. Compendium of Pharmaceutical and Specialties Thirty-fifth Edition (2000).

Toxicities due to cisplatin include cardiovascular toxicity such as electocardiographic changes; dermatologic toxicity such as hyperpigmentation; extravasation hazard toxicity such as irritant; gastrointestinal toxicities such as nausea and vomiting; hematologic toxicities such as myelosuppression and hemolytic anemia; hypersensitivity toxicity such as anaphylactic; neuromuscular toxicity such as peripheral neurophathy and acute encephalopathy; ocular toxicity such as retrobulbar neuritis; otologic toxicities such as hearing loss and tinnitus; renal/metabolic toxicities such as toxic nephropathy and hypokalemia; and, other toxicity such as infertility. This anti-proliferative agent has been used to treat several cancers including, for example, bladder, small cell lung, ovarian, testicular, brain, breast, cervical, head and neck, hepatoblastoma, and thyroid cancers. Compendium of Pharmaceutical and Specialties Thirty-fifth Edition (2000).

Toxicities due to gemcitabine hydrochloride include, for example, hematologic toxicities such as myelosuppression; gastrointestinal toxicities such as nausea, vomiting and stomatitis; hepatic toxicities such as transient elevations of serum transaminases; renal toxicities such as proteinuria, hematuria, hemolytic uremicsyndrome and renal failure; dermatologic toxicity such as rash and alopeica; edema toxicities such as edema and peripheral edema; and, other toxicity such as fever. This anti-proliferative agent has been used to treat pancreatic and non-small cell lung cancers. Compendium of Pharmaceutical and Specialties Thirty-fifth Edition (2000).

The present discussion comprises prevention or treatment of localized cancers or solid tumors that can be treated include those of the prostate, breast, pancreas, liver, kidney, genitourinary system, brain, gastrointestinal system, respiratory system, and head and neck. The compositions, etc., herein may prevent or treat cancers, including metastases, by allowing controlled release of fucan-complex at a site somewhat distant from the target tumors by allowing effective concentrations of the fucan-complex to reach the tumors and/or metastases by diffusion or even systemic transport. Some of these cancers are discussed further in the following paragraphs.

Prostate Cancer:

Prostate cancer is a malignant tumor that arises in the cells lining the prostate gland. In the U.S., an estimated 200,000 patients will develop prostate cancer this year, and more than 30,000 will die of the disease. Prostate cancer has a deaths to new cases ratio of ˜15%. The cancer may remain within the prostate, or it may spread to surrounding tissues or to distant sites (most often lymph nodes and bone). Usually prostate cancer spreads silently, producing symptoms only when it has progressed beyond the prostate. If prostate cancer has been diagnosed and treated during early stages, in some studies patients have had a 5-year survival rate of 94%.

Prostate cancer is often discussed as a disease of men over age 50. In fact, 80% of men with prostate cancer are 60 years of age and older. A man's chances of being diagnosed with prostate cancer during his lifetime are about 1 in 10, roughly the same as a woman's chances of having breast cancer. The number of reported new cases has risen dramatically in recent years as a result of improved tests that can detect the disease early in its development, often long before symptoms appear. The likelihood of developing prostate cancer in any given year increases with age, but rises dramatically after age 50.

Current treatment options for prostate cancer depend upon the extent of disease progression, the patient's age and overall health. Elderly patients, who have only early stage cancer or who suffer from additional, more serious diseases, may be treated conservatively, whereas those whose cancer is advanced may undergo more aggressive treatment. Prostate cancer has been treated by various methods, including radiation therapy (external beam radiation or brachytherapy), hormone withdrawal or castration (surgical or chemical), anti-proliferative agents, surgery, and expectant therapy (that is, “watchful waiting”). No treatment guarantees an absolute cure, and some have considerable side effects.

Early stage prostate cancer (that is, the tumor is localized to the prostate) may be treated with “watchful waiting”. Surgery for prostate cancer has been recommended for patients whose overall health has been otherwise good and the tumor is confined to the prostate gland. A common treatment for localized cancer of the prostate in men under the age of 70 has been radical prostatectomy (that is, surgical removal of the prostate).

Patients whose cancer is localized in the prostate area are commonly treated with external beam radiation (EBR). The radiation kills cancer cells and shrinks tumors. EBR accounts for less than 20% of localized prostate cancer treatment, with approximately 50% of these patients experiencing post radiation recurrences of the disease. Combined with early stage prostate cancer detection and increased demand from patients, brachytherapy (i.e., local radiation therapy) use has been expected to grow. In 1995, only 2.5% of newly diagnosed patients were treated using brachytherapy. Brachytherapy involves the implantation of radioactive metal “seeds” in the prostate tumor.

Treatment for prostate cancer that has spread involves removal of the testicles or hormone therapy. Both are used to inhibit or stop the production of the testosterone that has been driving the cancer growth. Approximately 20% of all prostate cancer patients undergo hormone withdrawal therapy. Hormone therapies include goserelin acetate (Zoladex®) or leuprolide acetate (Lupron®). Anti-proliferative agents used to treat prostate cancer have included 5-fluorouracil.

Breast Cancer:

In the U.S., breast cancer has been the most common cancer among women, with about 180,000 new cases diagnosed every year (male breast cancer accounts for about 5% of all diagnosed breast cancers). It has been surpassed only by lung cancer as a cause of death in women, and it has been responsible for approximately 50,000 deaths annually. An American woman has a one in eight (or about 13%) chance of developing breast cancer during her lifetime. Over the past decade, most reported breast cancers were small, primary (arising independently; not caused by a metastasis) tumors. Roughly 70% to 80% of newly diagnosed patients exhibited early-stage disease (Stage 1 or 2), and a majority had no involvement of the axillary (underarm) lymph nodes.

Most breast cancers are carcinomas (that is, malignant tumors that grow out of epithelial tissues). Less than 1% of breast cancers are sarcomas, or tumors arising from connective tissue, bone, muscle or fat. In addition, most breast cancers (about 75%) are ductal carcinomas, arising in the tissues that line the milk ducts. A much smaller number of cancers (about 7%) are found within the breast lobules and are called lobular carcinomas. Paget's disease (cancer of the areola and nipple) and inflammatory carcinoma account for nearly all other forms of breast cancer.

Breast cancer treatment has been complicated and depends on many factors. Two important factors are the type of tumor and the stage of progression. Tumor characteristics, in particular, help to separate individuals into two groups: (1) those who are at low risk of cancer recurrence and (2) those who are at high risk of cancer recurrence. Specific prognostic factors place patients in either of these groups.

These factors include tumor size; presence of female sex hormone estrogen and progesterone (ER/PR) receptors; cellular growth cycle phase (whether tumor cells are actively dividing or are in “S-phase”); presence of a protein known as “her-2-neu protein”; tumor grade, an indicator of tumor cell differentiation or change; and, tumor ploidy, the number of sets of genetic material within tumor cells.

Treatment of primary disease without significant lymph node involvement has been by lumpectomy and radiotherapy. More significant lymph node involvement may warrant mastectomy and removal of auxiliary lymph nodes. At this stage the chance of metastasis and local recurrence has been high. Treatment of metastatic disease has been palliative, involving radiation therapy and chemotherapy, which are immunosuppressive, cytotoxic and leukopoenic. Anti-proliferative agents including, for example, 5-fluorouracil, doxorubicin, methotrexate, and paclitaxel, have been approved for use against breast cancer.

Pancreatic Cancer:

The pancreas is an organ of the digestive system located near the stomach and small intestine. It has two major functions: the production of enzymes and hormones. Cancers of the pancreas can occur in the exocrine (i.e., enzymes) pancreas (e.g., classic pancreatic adenocarcinomas) or can occur in the endocrine (i.e., hormones) pancreas.

Cancers of the exocrine pancreas are a very serious health issue. In the U.S., approximately 28,000 patients are diagnosed with pancreatic cancer, while about the same number die annually from this disease. Pancreatic cancer occurs equally in males and females. Due to difficulties in diagnosis, the intrinsic aggressive nature of pancreatic cancers, and the sparse systemic treatment options available, only approximately 4% of patients diagnosed with pancreatic adenocarcinoma live for 5 years after diagnosis. Pancreatic cancer has been the 5^{th} leading cause of cancer death, following breast, lung, colon, and prostate cancer.

The choice of treatment for pancreatic cancer depends largely on the stage of the tumor. Possible treatments include surgery, anti-proliferative agents, radiation, and biological therapy. Surgery has been usually reserved for Stage 1 patients whose cancer is deemed resectable. Sometimes a combination of therapies, such as radiation and anti-proliferative agent given before or after surgery, can increase a patient's chances of survival. Pancreatic cancer that is deemed unresectable (usually Stage II or later) may be treated using anti-proliferative agents in clinical trials. Anti-proliferative agents, such as, for example, gemcitabine or 5-fluorouracil have had some effect against pancreatic cancer and gemcitabine has been used as a palliative agent. Toxicities due to these anti-proliferative agents are discussed elsewhere herein. Radiation therapy has some effect against pancreatic cancer when used in combination with chemotherapy. Radiation therapy alone may subdue symptoms. This form of treatment has also been used in Stage II or later pancreatic cancers.

Bladder Cancer:

In 1998, it was estimated that over 54,000 new cases of bladder cancer would be diagnosed in the U.S. and about 15,000 deaths would be attributed to the disease. Bladder cancer has been the fourth most common cancer among American men and the ninth most common cancer among American women. It occurs three times more frequently in men than in women. Primarily a disease of older men, bladder cancer has been a significant cause of illness and death. The risk of bladder cancer increases steeply with age (80% of cases occur in people older than 50 years), with over half of all bladder cancer deaths occurring after age 70. In white men over 65, the annual disease rate of bladder cancer has been approximately 2 cases per 1,000 persons; this contrasts with a rate of 0.1 cases per 1,000 persons under 65. During one's lifetime, the probability of developing bladder cancer has been greater than 3%; however, the probability of dying, from bladder cancer has been small (<1%). Bladder cancer rarely occurs in people who are younger than 40 years of age.

Recent studies suggest that certain genes and inherited metabolic abilities may play a role in bladder cancer. Transitional cell carcinoma (TCC) has been the most common form of bladder cancer. TCC usually occurs as a superficial (surface), papillary (wart-like), exophytic (outward-growing) mass upon a stalk-like base. In some cases, though, TCC may be attached on a broad base or it may appear ulcerated (within an indented lesion). Papillary TCCs often start out as areas of hyperplasia that later dedifferentiate, or lose individual cell characteristics. Only about 10% to 30% of papillary TCCs develop into invasive cancers. By contrast, nonpapillary forms of TCC are more likely to become invasive. As noted, such TCCs may appear ulcerated or flat. Flat, nonpapillary TCC that has been made up of anaplastic epithelium has been classified as carcinoma in situ (CIS or TIS). The tissue of CIS contains cells that are large, have noticeable nucleoli (round body within a cell; involved in protein synthesis), and lack normal polarity.

The treatment of bladder cancer depends upon many factors. The most important of these factors are the type of tumor that is present and its stage. Common treatments include transurethral resection (TUR), electrosurgery, laser surgery, intravesical therapy, anti-proliferative agents, surgical therapy, cystectomy, and radiation therapy. Examples of anti-proliferative agents used to treat bladder cancer include, for example, 5-fluorouracil, cisplatin and methotrexate. Toxicities due to the anti-proliferative agents, 5-fluorouracil, cisplatin, and methotrexate, are discussed elsewhere herein.

Brain Cancer:

Brain tumors are often inoperable and more than 80% of patients die within 12 months of diagnosis. Approximately 18,000 new cases of primary intracranial (brain) cancer are diagnosed each year in the U.S. This represents about 2 percent of all adult cancers. More than 50 percent of these are high-grade gliomas (i.e., glioblastoma multiform and anaplastic astrocytoma tumors). Patients with these tumors often suffer from severe disabilities such as motor dysfunction, seizures, and vision abnormalities.

Tumors that begin in brain tissue are known as primary brain tumors. Primary brain tumors are classified by the type of tissue in which they begin. The most common brain tumors are gliomas, which begin in the glial (supportive) tissue. Others include astrocytomas, brain stem gliomas, ependymomas and oligodendrogliomas.

Surgical removal of brain tumors has been recommended for most types and in most locations and should be as complete as possible within the constraints of preservation of neurologic function. An exception to this rule has been for deep-seated tumors, such as pontine gliomas, which are diagnosed on clinical evidence and are treated without initial surgery approximately 50% of the time. In many cases, however, diagnosis by biopsy is performed. Stereotaxic biopsy can be used for lesions that are difficult to reach and resect. Patients who have brain tumors that are either infrequently curable or unresectable should be considered candidates for clinical trials that evaluate radiosensitizers, hyperthermia, or interstitial brachytherapy used in conjunction with external-beam radiation therapy to improve local control of the tumor or for studies that evaluate new drugs and biological response modifiers.

Radiation therapy has a major role in the treatment of most tumor types and can increase the cure rate or prolong disease-free survival. Radiation therapy may also be useful in the treatment of recurrences in patients treated initially with surgery alone. Chemotherapy may be used before, during, or after surgery and radiation therapy. Recurrent tumors are treated with chemotherapy as well. Anti-proliferative agents used in the treatment of brain cancers include cisplatin. Examples of the toxicities associated with this anti-proliferative agent are discussed elsewhere herein.

### Restenosis

Restenosis is a form of chronic vascular injury leading to vessel wall thickening and loss of blood flow to the tissue supplied by the blood vessel. This inflammatory disease can occur in response to vascular reconstructive procedures including any manipulation that relieves vessel obstruction. Thus restenosis has been a major restrictive factor limiting the effectiveness of these procedures.

The present discussion comprises prevention or treatment of restenosis, for example by administering to a blood vessel a therapeutically effective amount of the combination of an oligonucleotide therapeutic and an anti-inflammatory agent. Suitable compositions include a polymeric carrier that can be surgically implanted at a restenosis site, or potential restenosis site, or can be injected via a catheter as a polymeric paste or gel.

### Arthritis

Rheumatoid arthritis (RA) is a debilitating chronic inflammatory disease characterized by pain, swelling, synovial cell proliferation (pannus formation) and destruction of joint tissue. In the advanced stage, the disease often damages critical organs and may be fatal. The disease involves multiple members of the immune system (macrophages/monocytes, neutrophils, B cells and T cells) complex cytokine interactions and synovial cell malfunction and proliferation. Early aggressive treatment has been recommended with disease modifying anti-rheumatic drugs (DMARDs) such as methotrexate, which drug is discussed elsewhere herein.

Crystal induced arthritis has been characterized by crystal induced activation of macrophages and neutrophils in the joints and is followed by excruciating pain for many days. The disease progresses so that the intervals between episodes gets shorter and morbidity for the patient increases. This disease has been generally treated symptomatically with non-steroidal anti-inflammatory drugs (NSAIDs) such as diclofenac sodium (Voltaren®). This anti-inflammatory agent has toxicities which include central nervous system toxicities such as dizziness and headache; dermatologic toxicities such as rash and pruritus; gastrointestinal toxicities such as exacerbated ulcerative colitis and Crohn's disease; genitourinary toxicities such as acute renal failure and renal papillary necrosis; hematologic toxicities such as agranulocytosis, leukopenia and thrombocytopenia; hepatic toxicities such as elevated liver transaminases and hepatitis; and, other toxicities such as asthma and anaphylaxis.

The methods herein, etc., prevent, treat or inhibit (similar to the effects on certain other diseases herein) rheumatoid arthritis, for example via administering to a patient a therapeutically effective amount of an oligonucleotide therapeutic and optionally an anti-inflammatory agent. Suitable compositions include a polymeric carrier that can be injected into a joint as a controlled release carrier of the anti-inflammatory agent and microparticulates as controlled release carriers of the oligonucleotide therapeutic (which in turn has been incorporated in the polymeric carrier). Such polymeric carriers may take the form of polymeric microspheres, pastes or gels.

### Inflammatory Conditions

The compositions, etc., herein may optionally inhibit or treat inflammatory conditions involving neutrophils for example comprising administering to a patient compositions containing an oligonucleotide therapeutic and an anti-inflammatory agent. Examples of such conditions include crystal-induced arthritis; osteoarthritis; non-rheumatoid inflammatory arthritis; mixed connective tissue disease; Sjögren's syndrome; ankylosing spondylitis; Behçet's syndrome; sarcoidosis; psoriasis; eczema; inflammatory bowel disease; chronic inflammatory lung disease; neurological disorders; and, multiple sclerosis. Some of these diseases are discussed further in the following paragraphs.

Inflammatory Bowel Disease (IBD):

This disease refers mainly to Crohn' s disease and ulcerative colitis that affect the intestine. IBD is an inflammatory disease characterized by periods of flare and remission. Joint inflammation may occur at the same time as a flare of IBD. Other complications of IBD may include inflammation of the skin, mouth, eye and may lead to cancer of the intestine. Chronic symptoms of this disease include intestinal blockage, perforation, abscess and bleeding. Symptoms may be treated with non-steroidal anti-inflammatory agents such as 5-aminosalicylic acid (Salofalk®). This anti-inflammatory agent has toxicities which include cardiovascular toxicity such as myocarditis; central nervous system toxicities such as headache and dizziness; gastrointestinal toxicities such as nausea and vomiting and diarrhea; genitourinary toxicities such as nephrotic syndrome and interstitial nephritis; hypersensitivity toxicities such as rash and pruritis; neuromuscular toxicity such as neuropathy; and, other toxicities such as hair loss and lichen planus.

Chronic Inflammatory Lung Diseases:

These inflammatory diseases include asthma, pneumoconiosis, obstructive pulmonary disease, nasal polyps and pulmonary fibrosis. Typically, such diseases are characterized by immune cell (such as neutrophils, macrophages and lymphocytes) activation and invasive inflammatory processes and thickening of the affected masses. Current drug therapies include the use of steroidal anti-inflammatory agents such as prednisone (Deltasone®). This anti-inflammatory agent has toxicities which include cardiovascular toxicities such as sodium and water retention; central nervous system toxicities such as headache, depression and convulsions; dermatologic toxicities such as impaired wound healing and acne; endocrine/metabolic toxicities such as menstrual irregularities and hypothalamic-pituitary-adrenal (HPA) axis suppression, Cushingoid appearance (e.g., moon faces, central obesity), growth suppression in children and osteoporosis; gastrointestinal toxicities such as peptic ulcer and pancreatitis; neuromuscular toxicity such as myopathy; ocular toxicities such as posterior subcapsular cataracts and glaucoma; and, other toxicities such as aseptic necrosis of femoral and humeral heads, spontaneous fractures and increased infection risk.

Chronic Inflammatory Skin Diseases (Including Psoriasis and Eczema):

Psoriasis is a common, chronic inflammatory skin disease characterized by raised, thickened and scaly lesions which itch, burn, sting and bleed easily. While these diseases have cellular proliferation and angiogenic components in later stages of the disease, patients often have accompanying arthritic conditions. Symptoms may be treated with steroidal anti-inflammatory agents such as prednisone or anti-proliferative agents such as methotrexate, which agents are discussed elsewhere herein.

The following provides some additional representative examples of inflammatory diseases that can be treated, etc., include, for example, arterial embolization in arteriovenous malformations (vascular malformations); menorrhagia; acute bleeding; central nervous system disorders; and, hypersplenism; inflammatory skin diseases such as psoriasis; eczematous disease (atopic dermatitis, contact dermatitis, eczema); immunobullous disease; and, inflammatory arthritis which includes a variety of conditions including rheumatoid arthritis, mixed connective tissue disease, Sjögren's syndrome, ankylosing spondylitis, Behçet's syndrome, sarcoidosis, crystal induced arthritis and osteoarthritis (all of which feature inflamed, painful joints as a prominent symptom).

Further representative diseases include inflammatory bowel disease (IBD) including ulcerative colitis and Crohn's disease; surgical adhesions; periodeontal disease; polycystic kidney disease; chronic inflammatory diseases of the respiratory tract including asthma, chronic obstructive pulmonary disease (COPD), chronic bronchitis, asthmatic bronchitis, chronic obstructive bronchitis, and emphysema and other diseases which lead to chronic airway obstruction; diseases associated with the obstruction of body passageways including, for example, vascular diseases, neoplastic obstructions, inflammatory diseases and infectious diseases; and, neovascular diseases of the eye including, for example, corneal neovascularization, neovascular glaucoma, proliferative diabetic retinopathy, retrolental fibroblasia and macular degeneration.

The compositions discussed herein can also be used to treat vascular diseases that cause obstruction of the vascular system. Such diseases include artherosclerosis of all vessels (around any artery, vein or graft) including, but not restricted to: the coronary arteries, aorta, iliac arteries, carotid arteries, common femoral arteries, superficial femoral arteries, popliteal arteries, and at the site of graft anastomosis; vasospasms (for example, coronary vasospasms and Raynaud's disease); restensosis (obstruction of a vessel at the site of a previous intervention such as balloon angioplasty, bypass surgery, stent insertion and graft insertion); inflammatory and autoimmune conditions (for example, temporal arteritis and vasculitis).

The compositions herein can be used for preventing or treating inflammatory diseases, acute or chronic, which affect or cause the obstruction of a body passageway. Representative examples include vasculitis (for example, giant cell arteritis (temporal arteritis and Takayasu' s arteritis), polyarteritis nodosa, allergic angiitis and granulomatosis (Churg-Strauss disease), polyangiitis overlap syndrome, hypersensitivity vasculitis (Henoch-Schonlein purpura), serum sickness, drug-induced vasculitis, infectious vasculitis, neoplastic vasculitis, vasculitis associated with connective tissue disorders, vasculitis associated with congenital deficiencies of the complement system, Wegener's granulomatosis, Kawasaki's disease, vasculitis of the central nervous system, Buerger's disease and systemic sclerosis; gastrointestinal tract diseases (for example, pancreatitis, Crohn's disease, ulcerative colitis, ulcerative proctitis, primary sclerosing cholangitis, benign strictures of any cause including idiopathic (for example, strictures of bile ducts, esophagus, duodenum, small bowel or colon)); respiratory tract diseases (for example, asthma, hypersensitivity pneumonitis, asbestosis, silicosis and other forms of pneumoconiosis, chronic bronchitis and chronic obstructive airway disease); nasolacrimal duct diseases (for example, strictures of all cases including ideopathic); and, Eustachian tube diseases (for example, strictures of all cases including ideopathic).

The compositions herein can also be used for treating or preventing infectious diseases associated with or causative of the obstruction of a body passageway. Briefly, infectious diseases include several acute and chronic infectious processes that can result in obstruction of body passageways including, for example, obstructions of the male reproductive tract (for example, strictures due to urethritis, epididymitis, prostatitis); obstructions of the female reproductive tract (for example, vaginitis, cervicitis, pelvic inflammatory disease (for example, tuberculosis, gonococcus, chlamydia, enterococcus and syphilis)); urinary tract obstructions (for example, cyctitis, urethritis); respiratory tract obstructions (for example, chronic bronchitis, tuberculosis, other mycobacterial infections (MAI, etc.), anaerobic infections, fungal infections and parasitic infections); and, cardiovascular obstructions (for example, mycoticaneurysms and infective endocarditis).

### Medical Device, Combination, and Pharmaceutical Products

The discussion herein also provides medical devices, combination, and pharmaceutical products, comprising compositions as discussed herein in a medical device, combination product or pharmaceutically acceptable container. The products can also include a notice associated with the container, typically in a form prescribed by a governing agency regulating the manufacture, use, or sale of medical devices, combination, and pharmaceuticals or biopharmaceuticals, whereby the notice is reflective of approval by the agency of the compositions, such as a notice that a fucan-dendrimer complex has been approved as an anti-proliferative agent or anti-inflammatory agent, e.g., for human or veterinary administration to treat proliferative diseases or inflammatory diseases (such as, for example, inflammatory arthritis, restenosis, surgical adhesions, psoriasis, graft rejections, inflammatory bowel disease, multiple sclerosis, and inflammatory lung disease). Instructions for the use of the fucan-dendrimer complex compositions herein may also be included. Such instructions may include information relating to the dosing of a patient and the mode of administration.

The present application is further directed to methods of making the various elements of the fucan-dendrimer complex compositions, systems etc., discussed herein, including making the compositions themselves, as well as to methods of using the same, including for example treatment of the conditions, diseases, etc., herein.

### EXAMPLES

### Example 1: Hydrogen Bonding

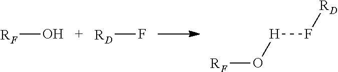

R_{F} is a fucan.

R_{D} is a dendrimer. The dendrimer may be a hyperbranched polyglycerol. The hyperbranched polyglycerol may be one of the hyperbranched polyglycerols discussed in references 8, 11, 12 or 18.

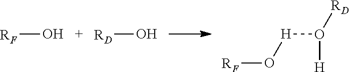

R_{F} is a fucan.

R_{D} is a dendrimer. The dendrimer may be a hyperbranched polyglycerol. The hyperbranched polyglycerol may be one of the hyperbranched polyglycerols discussed in references 1-25. The dendrimer may be a hyperbranched polyacrylate. The dendrimer may be a hyperbranched beta-cyclodextrin. The dendrimer may be a hyperbranched polyester.

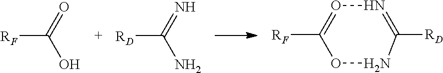

R_{F} is a fucan.

R_{D} is a dendrimer. The dendrimer may be a hyperbranched polyglycerol. The hyperbranched polyglycerol may be one of the hyperbranched polyglycerols described in references 1-4, 8, 9, 11-19, 20-25.

### Example 2: Covalent Linking Through a Methyl Sulfate Ester Intermediate

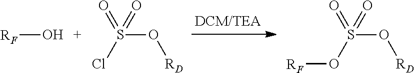

R_{F} is a fucan.

R_{D} is a dendrimer. The dendrimer may be a hyperbranched polyglycerol.

### Example 3: Covalent Linking Through a Thioglycol Linker to a Thiol Functional Group

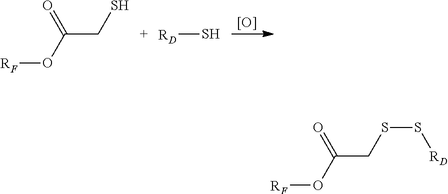

R_{F} is a fucan.

R_{D} is a dendrimer. The dendrimer may be a hyperbranched polyglycerol. The hyperbranched polyglycerol may be one of the hyperbranched polyglycerols discussed in references 3, 4, 8, 11, 12, 23, 25.

### Example 4a: Covalent Linking Through a Carboxylic Acid Functional Group on Fucoidan

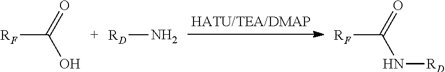

R_{F} is a fucan.

R_{D} is a dendrimer. The dendrimer may be a hyperbranched polyglycerol. The hyperbranched polyglycerol may be one of the hyperbranched polyglycerols discussed in references 1-4, 8, 9, 11-19, 20-25. The dendrimer may be a hyperbranched polyethylenemine. The dendrimer may be a hyperbranched polyamido amine

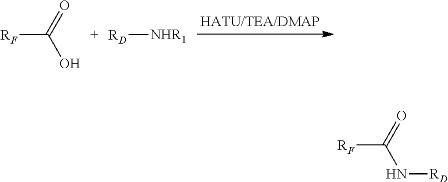

R_{F} is a fucan.

R_{D} is a dendrimer. The dendrimer may be a hyperbranched polyglycerol. The hyperbranched polyglycerol may be one of the hyperbranched polyglycerols discussed in references 1-4, 8, 9, 11-19, 20-25. The dendrimer may be a hyperbranched polyethylenemine. The dendrimer may be a hyperbranched polyamido amine

R₁ may be one of —H, —X, C₁₋₃₀ alkyl, C₁₋₃₀ allyl, C₁₋₃₀ alkenyl where any and all hydrogens may be independently substituted with X.

Where X═F, Cl, Br, I or OH.

### Example 4b: Covalent Linking Through a Carboxylic Acid Functional Group on Fucoidan

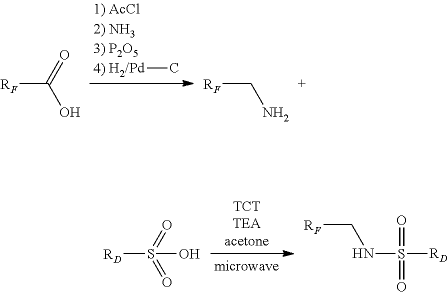

R_{F} is a fucan.

R_{D} is a dendrimer. The dendrimer may be a hyperbranched polyglycerol. The hyperbranched polyglycerol may be one of the hyperbranched polyglycerols discussed in references 1-3, 8, 11, 12 ,14-17, 20, 24.

### Example 5a: Covalent Linking Through Primary Hydroxyl Group on Fucoidan

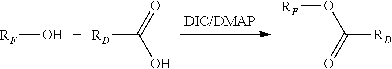

R_{F} is a fucan.

R_{D} is a dendrimer. The dendrimer may be a hyperbranched polyglycerol.

The hyperbranched polyglycerol may be one of the hyperbranched polyglycerols discussed in references 1-4, 8, 11, 12, 14-18, 20, 25.

### Example 5b: Covalent Linking Through Primary Hydroxyl Group on Fucoidan

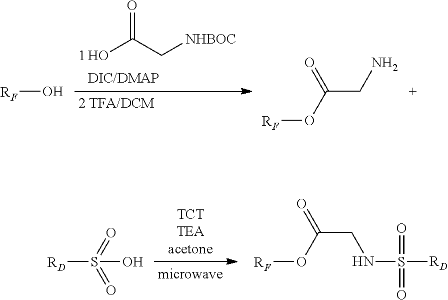

R_{F} is a fucan.

R_{D} is a dendrimer. The dendrimer may be a hyperbranched polyglycerol. The hyperbranched polyglycerol may be one of the hyperbranched polyglycerols discussed in reference 1-3, 8, 11, 12 ,14-17, 20, 24.

### Example 6: Covalent Linking Through Secondary Hydroxyl Group on Fucoidan

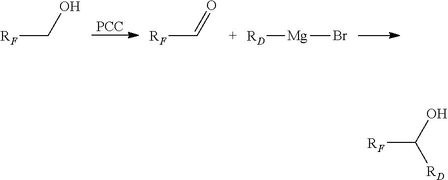

R_{F} is a fucan.

R_{D} is a dendrimer. The dendrimer may be a hyperbranched polyglycerol. The hyperbranched polyglycerol may be one of the hyperbranched polyglycerols discussed in references 1-4, 8, 11, 12, 14-18, 20, 25.

### Example 7: Co-Ordination Bond with Metal

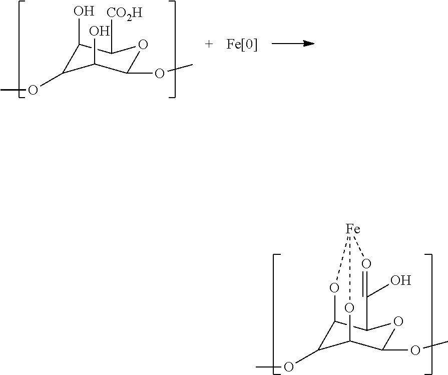

The iron ion may be present on a dendrimer. The dendrimer may be a hyperbranched polyglycerol.

### Example 8: Covalent Linking to a Sulfate Group on Fucoidan

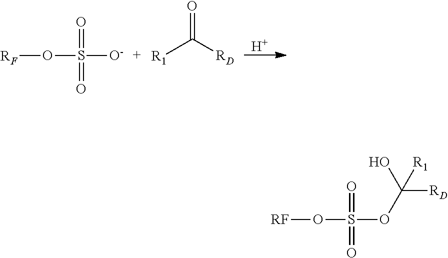

R_{F} is a fucan.

R_{D} is a dendrimer. The dendrimer may be a hyperbranched polyglycerol. The hyperbranched polyglycerol may be one of the hyperbranched polyglycerols discussed in references 1-4, 8, 11, 12, 14-18, 20, 25. The dendrimer may be a hyperbranched polyacrylate. The dendrimer may be a hyperbranched polyamido amine The dendrimer may be a hyperbranched polyester.

R₁ may be one of —H, —X, C₁₋₃₀ alkyl, C₁₋₃₀ allyl, C₁₋₃₀ alkenyl where any and all hydrogens may be independently substituted with X.

Where X═F, Cl, Br, I or OH.

### Example 9: Ionic Bonding to Cationic Groups

The sulfate group on a fucan may form an ionic bond with cationic groups on other complexes.

The cationic group may be present on a dendrimer. The dendrimer may be a hyperbranched polyglycerol. The hyperbranched polyglycerol may be one of the hyperbranched polyglycerols discussed in references 1-3, 8, 11, 12, 14-18, 20, 22, 23.

### Example 10: Magnetic Attachment to a Magnet

The fucan is functionalized to introduce a magnetic moiety. The magnetic moiety on the functionalized fucan may then form a magnetic attachment with a magnetic compound.

The magnetic compound may be a functionalized dendrimer. The dendrimer may be a functionalized hyperbranched polyglycerol.

### Example 11: Efficacy of Fucan-HPG Complex Formulation for the Treatment of Arthritis

The fucan may be covalently linked to a HPG through one of the following mechanisms:
i. Fucan with a carboxylic acid functional group may be attached to a primary amine functionality on a HPG as shown in example 4a.
ii. Fucan with a carboxylic acid functional group may be attached to a sulfite functionality on a HPG as shown in example 4b.
iii. Fucan may be attached to a carboxylic acid functionality on a HPG as shown in example 5a.
iv. Fucan may be attached to a sulfite functionality on a HPG as shown in example 5b
v. Fucan with a secondary alcohol functionality may be attached to a ketal or aldehyde group on a HPG as shown in example 6.
vi. Fucan may be attached to a ketal group on a HPG as shown in example 8.
vii. Fucan may be coordinated to a metal ion such as iron, wherein the iron is coordinated to a HPG.

The fucan-HPG complex is dissolved at between 0.1-850 mg/mL in Lactated Ringer's Injection USP. About 1-10 mL, of the fucan-HPG complex formulation placed in a 10 mL headspace vial. The vial is stoppered with a rubber septum and sealed with an aluminum crimp. Sterilization is accomplished by autoclave and the solution is allowed to cool to ambient temperature. The fucan-HPG complex formulation is used as is or is diluted in Lactated Ringer's Injection USP prior to injection. A person exhibiting the symptoms of arthritis receives between 0.1-5 mL, for example between 2-4 mL of the fucan-HPG complex formulation via intra-articular injection, locally at the site of arthritis. The site of the arthritis may be the knee joint. Following treatment with the fucan-HPG complex formulation, the symptoms of arthritis at the treated site may decrease in severity. The fucan may be fucoidan.

### Example 12: Efficacy of Timed Release Fucan-HPG Complex Formulation for the Prevention of Fibrous Adhesions

The fucan may be attached to HPG through one of the following mechanisms:
i. Hydrogen bonding between hydroxyl groups on the fucan and highly electronegative atoms on the HPG.
ii. Covalent binding to a carboxylic acid functionality on a HPG as shown in example 5a.
iii. Covalent binding to a sulfite functionality on a HPG as shown in example 5b.

The fucan-HPG complex formed contains bonds that allow a controlled release of fucan from the fucan-HPG complex. The fucan-HPG complex is dissolved at 0.1-850 mg/mL in Lactated Ringer's Injection USP. About 10 mL of the fucan-HPG complex formulation is placed in a 10 mL headspace vial. The vial is stoppered with a rubber septum and sealed with an aluminum crimp. Sterilization is accomplished by autoclave and the solution is allowed to cool to ambient temperature. The fucan-HPG complex formulation may be used as sterilized or diluted in Lactated Ringer's Injection USP. A desired volume of the fucan-HPG complex formulation is instilled at the surgical site post-surgery in the patient.

Following treatment with the fucan-HPG complex formulation, the surgical adhesions at the surgical site (or other target site) may be prevented from forming and/or may decrease in number and severity. The surgical site may be a peritoneal cavity. The number and severity of surgical adhesions at the surgical site (or other target site) may decrease for example, by between 30-100%.

In the case of (i), the disruption of hydrogen bonds of the fucan-HPG complex at the surgical site may result in a release of the fucan at the surgical site that typically starts at administration then proceeds generally linearly up to about 5, 10, 15, 20, 29 or 30 days, increasing the residence time of the fucan at the surgical site compared to unbound fucan, which may typically clear from the site in about 1, 2, 3 or 5 days. This can improve the efficacy of prevention of surgical adhesion and may also allow the use of lower fucan dose due to increased residence time.

In the case of (ii) and (iii) the cleavage of covalent linkages between the fucan and HPG in the fucan-HPG complex at the surgical site may result in a release of the fucan at the surgical site that typically starts then proceeds generally linearly up to 100, 200, 300 or 500 days. This increases the residence time for the fucan at the surgical site.

In certain embodiments, a combination of both fucan hydrogen bonded to HPG (or other dendrimer) and fucan covalently bonded to HPG (or other dendrimer) is provided in a single composition to provide a composition, with both short-term controlled release and long-term controlled release of the fucan from the HPG. Further, compositions herein include a combination of fucan unattached to a secondary component, which will typically clear the target site in about 1, 2, 3 or 5 days, plus a fucan hydrogen bonded to HPG (or other dendrimer), which will typically clear the target site in about 5, 10, 15, 20, 29 or 30 days, plus a fucan covalently bonded to HPG (or other dendrimer), which may typically clear the target site in about 100, 200, 300 or 500 days.

Fucan is known to increase APTT and decrease blood coagulation at some doses. Increased APTT and decreased blood coagulation is a side effect resulting from the use of fucan in the prevention of fibrous adhesions at some doses. The resulting slow release of fucan from fucan-HPG complexes may reduce these side effects. The resulting slow release of fucan from fucan-HPG complexes may result in a decrease in APTT spiking by between 10-100%.

### Example 13: Efficacy of Fucan-HPG Complex Formulation for the Treatment of Prevention of Fibrous Adhesions

The fucan may be covalently linked to a HPG through one of the following mechanisms:
viii. Fucan with a carboxylic acid functional group may be attached to a primary amine functionality on a HPG as shown in example 4a.
ix. Fucan with a carboxylic acid functional group may be attached to a sulfite functionality on a HPG as shown in example 4b.
x. Fucan may be attached to a carboxylic acid functionality on a HPG as shown in example 5a.
xi. Fucan may be attached to a sulfite functionality on a HPG as shown in example 5b
xii. Fucan with a secondary alcohol functionality may be attached to a ketal or aldehyde group on a HPG as shown in example 6.
xiii. Fucan may be attached to a ketal group on a HPG as shown in example 8.
xiv. Fucan may be coordinated to a metal ion such as iron, wherein the iron is coordinated to a HPG.

The fucan-HPG complex formed may have increased mucoadhesive properties. The fucan-HPG complex is dissolved at 0.1-850 mg/mL in Lactated Ringer's Injection USP. About 10 mL of the fucan-HPG complex formulation is placed in a 10 mL headspace vial. The vial is stoppered with a rubber septum and sealed with an aluminum crimp. Sterilization is accomplished by autoclave and the solution is allowed to cool to ambient temperature. The fucan-HPG complex formulation may be used as sterilized or diluted in Lactated Ringer's Injection USP. A desired volume of the fucan-HPG complex formulation is instilled at the surgical site post-surgery in the patient is instilled at the surgical site post-surgery.

Following treatment with the fucan-HPG complex formulation, the surgical adhesions at the surgical site (or other target site) may be prevented from forming and/or may decrease in number and severity. The surgical site may be a peritoneal cavity. The number and severity of surgical adhesions at the surgical site (or other target site) may decrease for example, by between 30-100%.

The fucan may be fucoidan.

### LIST OF REFERENCES

1. Brooks E., D., Kizhakkedathu N., J., & Kainthan Kumar, R. (2008). United States Patent No. 20080292579.
2. Brooks E., D., Kizhakkedathu N., J., & Kainthan Kumar, R. (2016). United States Patent No. 20170042804.
3. Burt M., H., Donald, B., Jayachandran, K., Richard, L., Dechi, G., Lu, Y., . . . Ala, S. (2013). United States Patent No. 20130122112.
4. Calderon, M., Welker, P., Licha, K., Fichtner, I., Graeser, R., Haag, R., & Kratz, F. (2011). Development of efficient acid cleavable multifunctional prodrugs derived from dendritic polyglycerol with a poly(ethylene glycol) shell. Journal of Controlled Release, 295-301.
5. Du, C., Mendelson, A. A., Guan, Q., Chapanian, R., Chafeeva, I., Roza, G. d., & Kizhakkedathu, J. N. (2014). The size-dependent efficacy and biocompatibility of hyperbranched polyglycerol in peritoneal dialysis. Biomaterials, 1378-1389.
6. Du, C., Mendelson, A. A., Guan, Q., Dairi, G., Chafeeva, I., Roza, G. d., & Kizhakkedathu, J. N. (2016). Hyperbranched polyglycerol is superior to glucose for long-term preservation of peritoneal membrane in a rat model of chronic peritoneal dialysis. Journal of translational medicine.
7. Garamus, V. M., Maksimova, T. V., Kautz, H., Barriau, E., Frey, H., Schlotterbeck, U., . . . Richtering, W. (2004). Hyperbranched Polymers: Structure of Hyperbranched Polyglycerol and Amphiphilic Poly(glycerol ester)s in Dilute Aqueous and Nonaqueous Solution. Macromolecules, 8394-8399.
8. Helen, B. M., Brooks Donald, E., Jayachandran, K. N., Richard, L., Dechi, G., Lu, Y., & Clement, M. (2016). United States Patent No. 20170319696.
9. Imran ul-haq, M., Lai, B. F., & Kizhakkedathu, J. N. (2014). Hybrid Polyglycerols with Long Blood Circulation: Synthesis, Biocompatibility, and Biodistribution. Macromolecular Bioscience, 1469-1482.
10. Imran ul-haq, M., Shenoi, R. A., Brooks, D. E., & Kizhakkedathu, J. N. (2013). Solvent-Assisted Anionic Ring Opening Polymerization of Glycidol: Toward Medium and High Molecular Weight Hyperbranched Polyglycerols. Journal of Polymer Science, 2614-2621.
11. Jayachandran, K., Caigan, D., Donald, B., & Christopher, N. (2016). United States Patent No. 20160295856.
12. Jayachandran, K., Caigan, D., Roza, D. G., & Asher, M. (2015). United States Patent No. 20150141512.
13. Kainthan, R. K., Muliawan, E. B., Hatzikiriakos, S. G., & Brooks, D. E. (2006). Synthesis, Characterization, and Viscoelastic Properties of High Molecular Weight Hyperbranched Polyglycerols. Macromolecules, 7708-7717.
14. Kizhakkedathu N., J., Shenoi A., R., Carter J., C., & Brooks E., D. (2012). United States Patent No. 20120308546.
15. Kizhakkedathu N., J., Shenoi A., R., Carter J., C., & Brooks E., D. (2012). United States Patent No. 20130022647.
16. Kizhakkedathu N., J., Shenoi A., R., Carter J., C., & Brooks E., D. (2015). United States Patent No. 20150224244.
17. Kizhakkedathu N., J., Shenoi A., R., Carter J., C., & Brooks E., D. (2015). United States Patent No. 20150283171.
18. Kurniasih, I. N., Keilitz, J., & Haag, R. (2015). Dendritic nanocarriers based on. Chem Soc Rev, 4145-4164.
19. Liu, Z., Janzena, J., & Brooks, D. E. (2010). Adsorption of amphiphilic hyperbranched polyglycerol derivatives onto human red blood cells. Biomaterials, 3364-3373.
20. Michael, A., Podor J., T., Brooks E., D., & Kainthan K., R. (2010). United States Patent No. 20100324150.
21. Mugabe, C., Liggins, R., Guan, D., Manisali, I., Chafeeva, I., Brooks, D., . . . Burt, H. (2011). Development and in vitro characterization of paclitaxel and docetaxel loaded into hydrophobically derivatized hyperbranched polyglycerols. International Journal of Pharmaceutics, 238-249.
22. Shenoi, R. A., Lai, B. F., & Kizhakkedathu, J. N. (2012). Synthesis, Characterization, and Biocompatibility of Biodegradable Hyperbranched Polyglycerols from Acid-Cleavable Ketal Group Functionalized Initiators. Biomacromolecules, 3018-3030.
23. Wen, J., Weinhart, M., Lai, B., Kizhakkedathu, J., & Brooks, D. E. (2016). Reversible hemostatic properties of sulfabetaine/quaternary ammonium modified hyperbranched polyglycerol. Biomaterials, 42-55.
24. Wilms, D., Sritiba, S.-E., & Frey, H. (2010). Hyperbranched Polyglycerols: From the Controlled Synthesis of Biocompatible Polyether Polyols to Multipurpose Applications. Accounts of chemical research, 129-141.
25. Ye, L., Letchford, K., Heller, M., Liggins, R., Guan, D., Kizhakkedathu, J. N., . . . Burt, H. M. (2011). Synthesis and Characterization of Carboxylic Acid Conjugated, Hydrophobically Derivatized, Hyperbranched Polyglycerols as Nanoparticulate Drug Carriers for Cisplatin. Biomacromolecules, 142-155.

All terms used herein are used in accordance with their ordinary meanings unless the context or definition clearly indicates otherwise. Also unless expressly indicated otherwise, in the specification the use of “or” includes “and” and vice-versa. Non-limiting terms are not to be construed as limiting unless expressly stated, or the context clearly indicates, otherwise (for example, “including,” “having,” and “comprising” typically indicate “including without limitation”). Singular forms, including in the claims, such as “a,” “an,” and “the” include the plural reference unless expressly stated, or the context clearly indicates, otherwise.

Unless otherwise stated, adjectives herein such as “substantially” and “about” that modify a condition or relationship characteristic of a feature or features of an embodiment, indicate that the condition or characteristic is defined to within tolerances that are acceptable for operation of the embodiment for an application for which it is intended.

The scope of the present devices, systems and methods, etc., includes both means plus function and step plus function concepts. However, the claims are not to be interpreted as indicating a “means plus function” relationship unless the word “means” is specifically recited in a claim, and are to be interpreted as indicating a “means plus function” relationship where the word “means” is specifically recited in a claim. Similarly, the claims are not to be interpreted as indicating a “step plus function” relationship unless the word “step” is specifically recited in a claim, and are to be interpreted as indicating a “step plus function” relationship where the word “step” is specifically recited in a claim.

From the foregoing, it will be appreciated that, although specific embodiments have been discussed herein for purposes of illustration, various modifications may be made without deviating from the spirit and scope of the discussion herein. Accordingly, the systems and methods, etc., include such modifications as well as all permutations and combinations of the subject matter set forth herein and are not limited except as by the appended claims or other claim having adequate support in the discussion and figures herein.

What is claimed is:

1. A fucan-dendrimer complex comprising a fucan molecule attached to a dendrimer.

2. The fucan complex of claim 1 wherein the attachment is at least one of a covalent bond or a hydrogen bond.

3. The fucan complex of claim 1 wherein the attachment is at least one of an ionic bond, adsorption, dipole-dipole interaction, co-ordination complex formation or magnetic interaction.

4. The fucan-dendrimer complex of claim 1 wherein the dendrimer is a hyperbranched polyglycerol.

5. The fucan-dendrimer complex of claim 4 wherein the hyperbranched polyglycerol comprises at least one of a C₁-C₂₀ alkyl epoxide, C₁-C₂₀ alkyl glycidyl ether, glycerol epoxide, a C₁-C₂₀ alkyl.

6. The fucan-dendrimer complex of claim 4 wherein the hyperbranched polyglycerol comprises at least one of C₁-C₂₀ alkyl substituted with at least one OR group where R is one of a hydrogen, a cationic moiety, or a polymer segment.

7. The fucan-dendrimer complex of claim 1 wherein the dendrimer comprises at least one further substituent attached to the dendrimer.

8. The fucan-dendrimer complex of claim 7 wherein the further substituent on the dendrimer comprises at least one of amine, ammonium, thiol, sulfonic acid, phosphonic acid, carboxylic acid, tosyl, hydroxyl, N-hydroxysuccinimide, methoxy polyethylene glycol, polyethylene glycol, aryl group, or alkyl group.

9. The fucan-dendrimer complex of claim 7 wherein the further substituent is a biologically active moiety attached to the dendrimer.

10. The fucan-dendrimer complex of claim 1 wherein the dendrimer comprises a co-polymer.

11. The fucan-dendrimer complex of claim 10 wherein the co-polymer is at least one of polyglycerol, a polyol, a polyalkylene glycol, a polyalkylene glycol-alkyl ether, or an alkyl chain.

12. The fucan-dendrimer complex of any one of claims 1 to 11 wherein the fucan is fucoidan.
